(19) United States
(12) Patent Application Publication
(10) Pub. No.: US 2023/0356217 A1
(43) Pub. Date: Nov. 9, 2023

(54) A BLOOD GAS ANALYZER AND SYSTEM COMPRISING A BLOOD GAS ANALYZER, AND USE THEREOF

(71) Applicant: RADIOMETER MEDICAL APS, Brønshøj (DK)

(72) Inventors: Thea Ubbe EBBESEN, Brønshøj (DK); Ida Kjaergaard BAUMGARTEN, Brønshøj (DK)

(21) Appl. No.: 18/004,466

(22) PCT Filed: Jul. 9, 2021
(86) PCT No.: PCT/EP2021/069139

§ 371 Date: Jan. 6, 2023

(30) Foreign Application Priority Data
Jul. 10, 2020 (EP) 20185273.8

(51) Int. Cl. B01L 3/00; G01N 33/49

(52) U.S. Cl. CPC B01L 3/502715; G01N 33/4925; B01L 2200/025; B01L 2200/143; B01L 2200/18; B01L 2300/14; B01L 2400/049; B01L 2300/0636; B01L 2300/027

(57) ABSTRACT

A blood gas analyzer (1) for performing a measurement on analyte parameters in a blood sample, such as a whole blood sample, aspirated into the blood gas analyzer from a blood sample container comprises an output device, such as a monitor (30), for outputting instructions to a user of the blood gas analyzer for the user's handling of the one or more of the user-accessible parts of the blood gas analyzer, such as for maintenance purposes, and/or for replacement of sensor cassettes, gaskets, probes, solution packs, etc. A controller (8) is provided for receiving a signal, on the basis of which it is determined if an instruction, such as an animated video instruction, is to be presented to the user. In the affirmative, the controller selects one of a plurality of pre-stored sets of instructions for outputting at the output device.

### FIELD OF THE INVENTION

The present invention relates to a blood gas analyzer and a system for performing a measurement on analyte parameters in a blood sample, such as a whole blood sample. More specifically, the invention relates to improvements of a user interface of the blood gas analyzer for facilitating users' interaction with the blood gas analyzer with a view to increasing safety and ease of use.

### BACKGROUND OF THE INVENTION

Blood gas analyzers for measuring physical parameters of analytes in a blood sample by means of analyte sensors are widely used in the medical and clinical industry. Blood gas analyzers for use with a blood sample container typically comprise an aspiration point for making contact with a forward end of the handheld blood sample container. Once a fluid flow path has been established between the blood sample container and interior flow conduits of the blood gas analyzer, the blood sample or a portion thereof may be aspirated to the analyte sensors. Fluid handling infrastructures of blood gas analyzers typically include a number of reservoirs, consumables in the form of so-called ‘solution packs’ pre-filled with process liquids for, e.g., rinsing or wash-out, calibration and quality control tasks. Supply of an adequate process liquid for a given process step to a measurement chamber of a blood gas analyzer may be controlled by a valve system. Other consumables may include replaceable sensors, sensor cassettes, and inlet components that require intermittent or regular replacement.

Maintenance of blood gas analyzers, including repair and replacement of wearing parts and replacement of consumables such as, e.g., solution packs and sensor cassettes is necessary. In the context of point-of-care measurement systems (in the art also referred to as ‘bedside’ systems) and laboratory environments alike, maintenance tasks are done less frequently than blood sample analysis (typically once a month to once a year) and will often be carried out by trained lab technicians. Due to the task frequency, however, even trained users may forget how to carry out maintenance task of the blood gas analyzer. In some cases, maintenance tasks can be undertaken by untrained users, such as nurses, who may lack experience in carrying out maintenance tasks. Maintenance may thus not only constitute a nuisance to users, but may also cause blood gas analyzer down time, i.e. intermittent in-operational, and hence compromise efficiency in environments in which users rarely have time to waste.

Despite the availability of user manuals and training programs for users, a need exists for improved user guidance in the context of maintenance, notably with a view to reducing nuisance to users and blood gas analyzer down time and increasing efficiency.

### DESCRIPTION OF THE INVENTION

On the above background, it is an object of embodiments of the invention to provide a blood gas analyzer which facilitates users' interaction therewith, notably as far as the performance of maintenance tasks is concerned. It is a further object of embodiments of the invention to provide a blood gas analyzer, the use of which provides improved convenience to all users irrespective of their level of expertise and experience in carrying out maintenance tasks.

In a first aspect, the invention provides a blood gas analyzer for performing a measurement on analyte parameters in a blood sample aspirated into the blood gas analyzer from a blood sample container, the blood gas analyzer comprising a plurality of user-accessible parts, a controller and:
an aspiration system for aspirating the blood sample from the blood sample container, the aspiration system comprising an inlet structure for connecting to the blood sample container;
a monitor for outputting instructions to a user of the blood gas analyzer for the user's handling of the one or more of the user-accessible parts of the blood gas analyzer;
an electronic memory comprising at least two pre-stored sets of animated video instructions, each of the pre-stored sets of animated video instructions illustrating the user's correct handling of at least one of the user-accessible parts of the blood gas analyzer;
   wherein the controller is configured to:
receive a signal indicative of at least one of:
   a user input;
   a presence, a position and/or an orientation of the user-accessible parts, and
   at least one projected handling action of at least one of the user-accessible parts of the blood gas analyzer;
in response to the signal, determine if one of the animated video instructions is to be presented to the user, and in the affirmative:
   select at least one of the at least two pre-stored sets of animated video instructions on the basis of the signal; and
   output the at least one selected animated video instruction at the monitor.

The blood sample may, e.g., be a whole blood sample. In the present context the term ‘whole blood sample’ should be interpreted to be a sample of blood with all its components, including red and white blood cells, platelets and plasma. The blood may originate from human beings or animals, such as mammals.

The controller's ability to select at least one of a plurality of pre-stored sets of instructions in response to a signal as defined above provides a way of presenting instructions to the user, which guides the user to achieve convenient and efficient handling of the at least one user-accessible part. The instructions may, in particular, reflect workflows to be carried out by the user in respect of the handling action in question, i.e. a plurality of sequential steps. The handling action, which may be considered a maintenance action, may be defined by, or chosen in accordance with, the user input, the presence, position and/or orientation of the user-accessible parts, and/or by any information available to the controller indicative of the at least one projected handling action of the at least one of the user-accessible parts. Thus, for example, the controller may automatically select a set of instructions guiding the user through applicable steps of any maintenance or handling action due, as derived, e.g., through a user interface or communication interface to an external device, a sensor input, a maintenance schedule, or through any other data or signal indicating a need or a desire for the performance of a handling action.

The selection of the at least one of the plurality of pre-stored sets of instructions may advantageously be performed on the basis of the current behavior of the user, i.e. based on what is happening while the present interaction between the user and the analyzer is taking place, rather than based on information regarding the user during previous interactions. This allows a simple system, since access to historical data is not required. Furthermore, it can be ensured that the offered assistance matches the needs of the user under the given circumstances, such as the actual task being performed, the time of day, etc.

One advantage of the invention is the ability to adapt to the users' real time action and behavior. Adaptive guidance based on users' real time behavior creates value, as the same person's sample handling method may vary or differ depending of the situation and context. For instance, a person's handling may be different or deviate from normal in a stressful and critical situation, where a patient's life is depending of a fast result, compared with a non-critical situation.

Each set of instructions preferably defines a sequence of steps, i.e. a workflow or parts thereof, such as one or more steps thereof. The controller may be configured to determine, on the basis of the signal, if one or more the steps of each set of instructions is to be omitted from outputting at the output device. Thus, if for example, a set of instructions relates to the replacement of an assembly of elements defined by a series of steps for replacement of each of the elements, then if not all of such elements are due for exchange, the controller may select to output only those instructions or those parts of the instructions which relate to the elements to be replaced. Alternatively or additionally, if for example, a set of instructions relates to replacement of a user-accessible part defined by a series of five consecutive steps, and the controller receives a signal indicative of removal of the user-accessible part corresponding to step two in the sequence, i.e. a signal that step two has been completed, the controller will determine not to display the animated video instructions relating to step two.

The controller selecting at least one of the at least two pre-stored sets of animated video instructions on the basis of the signal, may comprise a controller assessment of signal, e.g., comprising comparing the received signal indicative of a presence, a position and/or an orientation of the user-accessible parts to a predefined threshold value, and select one of the pre-stored set of instructions depending on whether the received signal is above or below a predefined threshold value. For instance, in case the blood gas analyzer is handled by an experienced user, the received sensor signal may, e.g., be above the predefined threshold value. In case a novice user is handling the blood gas analyzer, the received sensor signal may, e.g., be below the predefined threshold value. In both cases a corresponding one of the pre-stored sets of instructions may be selected, and the instruction output device may accordingly output the selected one of the at least two sets of pre-stored sets of animated video instructions.

In order to adapt the presentation of instructions to the user to different levels of expertise of the user, the at least two pre-stored sets of animated instructions may comprise instructions of different level of detail. For example, a first one of the at least two sets of pre-stored sets of instructions may comprise instructions at a first level of detail for the user's interaction with the blood gas analyzer, and a second one of the at least two sets of pre-stored sets of instructions may comprise instructions at a second level of detail for the user's interaction the blood gas analyzer, the first level of detail being greater than the second level of detail.

On the one hand, the first level of detail may, for example, be tailored to match the needs of novice users which are not perfectly acquainted with the maintenance and/or calibration process of the blood gas analyzer. Such users may have a need for more elaborate help with handling the blood gas analyzer and/or its interaction with the user-assessable parts. Hence, the pre-stored sets of instructions, e.g., comprise detailed instructions on how to replace a user-accessible solution pack with an animated video instruction corresponding to each step of the process.

In a preferred embodiment, the output device includes a monitor, and the sets of instructions include animated video instructions. In the present context, the term ‘animated video sequences’ should be interpreted to mean a sequence in which visual elements are manipulated to appear as moving images. Animations may be made with computer-generated imaginary. The illusionary effect of animation may be achieved by a rapid succession of sequential images that minimally differ from each other. For instance, the animated video sequence may show how to correctly mount a ware part onto the blood gas analyzer's inlet structure. Further, animated video sequences may be generated to enhance the user's perception of any specific action and/or a haptic feedback to the user associated with any such action, and to illustrate the envisaged action from various perspectives. The animated video sequences may be generated to resemble to actual situation to which the user is presented, i.e. look like the task at hand. As a supplement to the animated videos, still pictures and/audio may form part of at least one of the at least two pre-stored sets of animated videos. In a fourth aspect, at least one of the pre-stored sets of animated video instructions is replaced by a pre-stored set of still pictures and/or audio clips.

Whilst in a preferred embodiment of the invention, the output device comprises a monitor, and the sets of instructions comprise instructions, such as animated video instructions in particular, other types of output devices and instructions are envisaged and within the scope of the present invention. For example, the instructions may comprise voice instructions, in which case the output device may comprise a loudspeaker. The instructions may alternatively or be provided by an external device, such as a tablet or smartphone, by means of which the instructions are provided to the user. In that case, the instructions may be stored at such external device, which outputs the relevant set of instructions or part thereof in response to data communicated to it through a communication interface of the blood gas analyzer.

Alternatively, the instructions may be stored in the electronic memory of the blood gas analyzer and communicated to the external device through the communication interface.

In the present context, the term user-accessible part is to be construed broadly to encompass any element included in or connectable to the blood gas analyzer, which is accessible by the user, be it directly, i.e. immediately accessible without any need for opening or dismounting covers, doors, panels, drawers or the like, or indirectly, i.e. accessible only after opening or removing a cover, door, panel, drawer or the like.

The user-accessible parts of the blood gas analyzer may comprise at least one of:
a solution pack,
an inlet module, such as an inlet module comprising the inlet structure, wherein said inlet structure is optionally comprising an inlet probe and/or inlet gasket with holder,
a sensor cassette, such as a replaceable unit for holding one or more analyte sensors,
an inlet probe of the inlet structure,
an inlet connector gasket, such as an inlet connector gasket being part of the blood gas analyzer interface stationary part,
an inlet gasket with a holder therefor.

The signal may be indicative of a need or a user desire to conduct at least one of:—
a manual flush for removing clots in a conduit of the blood gas analyzer,
replacement or installation of the solution pack,
replacement or installation of the sensor cassette,
replacement of the inlet probe,
replacement of the inlet connector gasket,
replacement of the inlet gasket with holder, and
replacement of the inlet module.

In order to provide information to a user regarding steps to be followed when correctly handling the at least one user-accessible part, each of the animated video instructions may illustrate a sequence of steps of the user's correct handling of the at least one user-accessible part. It is encompassed that the handling of at least one user-accessible part involves handlings of a plurality of user-accessible parts, optionally including both one or more immediately user-accessible parts and one or more indirectly user-accessible parts. For example, the sequence of steps may contain information about correct handling of both a user-accessible part, which is directly related to the purpose of the handling (for example a solution pack to be replaced), and additionally contain information about correct handling of user-accessible parts, which are merely indirectly related to the purpose of the handling (for example a lid which needs to be opened to allow user-access to the solution pack to be replaced and/or for example tubing which has to be disconnected before removing a solution pack to be discarded and connected to a new solution pack to be installed).

It may be advantageous to omit outputting information relating to completed steps, e.g., by having a blood gas analyzer wherein:
the signal indicative of the projected handling action includes an indication if one or more of said steps have been completed; and
the controller is configured to carry out the selection of the animated video instructions in such a manner that those one or more of the animated video instructions or those one or more parts thereof associated with the completed steps are omitted from outputting at the monitor.

In an example, the animated video instructions illustrate the user's correct handling of an inlet gasket with holder to be replaced and a replacement inlet gasket with holder, and upon completion of the step of removing the inlet gasket with holder to be replaced from the blood gas analyzer, the animated video instructions relating to said step is omitted from outputting at the monitor and instead subsequent steps, e.g., relating to installation of the replacement inlet gasket with holder can be outputted. An advantage thereof may be increased time-efficiency because the user is not presented with obsolete information which has to pass before the relevant information is outputted. Another advantage may be increased safety and/or improved ease of use because the fit between information presented and the actual situation of the blood gas analyzer is improved.

It may be advantageous to additionally or alternatively omit outputting information relating to steps beyond a current step, e.g., by having a blood gas analyzer wherein:
the signal indicative of the projected handling action includes an indication if a current step has been completed; and
the controller is configured to carry out the selection of the animated video instructions in such a manner that those one or more of the animated video instructions or those one or more parts thereof associated with subsequent steps are omitted from outputting at the monitor.

In an example, the animated video instructions illustrate the user's correct handling of an inlet gasket with holder to be replaced and a replacement inlet gasket with holder, and before completion of the step of removing the inlet gasket with holder to be replaced from the blood gas analyzer, the animated video instructions relating to subsequent steps are omitted from outputting at the monitor so that subsequent steps, e.g., relating to installation of the replacement inlet gasket with holder are not outputted until they have to be carried out by the user. An advantage thereof may be increased time-efficiency because the user is not presented with premature information. Another advantage may be increased safety and/or improved ease of use because the fit between information presented and the actual situation of the blood gas analyzer is improved.

An interaction between the user and the blood gas analyzer may involve haptic feedback to the user, e.g., wherein each of the pre-stored sets of animated video instructions comprises a simulation of a haptic feedback during the user's handling of the at least one of the user-accessible parts. Haptic feedback may be passive, for example where an animated video instruction shows an exaggerated illustration of overcoming a resistance, e.g., by displaying an animated hand or person putting in weight to apply a substantial force for overcoming a resistance, e.g., for pulling out an inlet gasket with holder. Alternatively, haptic feedback may be active, e.g., involving vibration or force feedback, for example where an animated video instruction shows vibration of a user-accessible part.

Thus, in the present context the term ‘simulation of a haptic feedback’ should be interpreted to mean that the animated video instruction indicates that the user is supposed to feel or experience a certain haptic feedback during performance of a given step or action. For instance, in the case that a user is supposed to feel a resistance when pushing or pulling a user accessible part, then the part of the animated video instructions which illustrate this step of pushing or pulling the accessible part also indicates the resistance which the user is supposed to feel. Thereby the user can be reassured that the manipulation of the accessible part is performed in a correct manner when the shown haptic feedback is in fact felt.

Similarly, in the case that a user is supposed to feel an active haptic feedback, such as a vibration or a force feedback, when performing a certain action, then the part of the animated video instructions which illustrates this action may also show the expected active haptic feedback, e.g. in the form of an exaggerated vibration of the part which provides the haptic feedback. Again, the user can be reassured that the action is performed in a correct manner when the shown haptic feedback is in fact felt.

The aspiration system may comprise an aspiration point for establishing a fluid flow communication with the blood sample container, and wherein the blood gas analyzer further comprises a detection structure for detecting a presence of the blood sample container at the aspiration point. The controller may furthermore be configured to:
deny the performance of analyte parameter measurements if it is determined that one or more of the at least one projected handling action or one more steps thereof is due; or to
prompt for a user confirmation of the performance of analyte parameter measurements if it is determined that one or more of the at least one projected handling action is due.

If it is determined that one or more of the at least one projected handling action or one or more steps thereof is due, it may be relevant to prompt for a user confirmation of the performance of analyte parameter measurements or even deny the performance of analyte parameter measurements, even if a detection structure has detected a presence of the blood sample container at the aspiration point. For example a user may provide a blood sample container at the aspiration point, but be denied (and optionally informed accordingly) the performance of analyte parameter measurements if it is determined that one or more of the at least one projected handling action or one more steps thereof is due. This may be advantageous since it eliminates or reduces one or more risks of, e.g., damage of the blood gas analyzer, malfunction of the blood gas analyzer and/or imprecise or erroneous analyte parameter measurements, which could in a worst case scenario be realized in case a user (knowingly or unknowingly) continued the performance of analyte parameter measurements even past a point where at least one projected handling action or one more steps thereof were due.

The signal indicative of the projected handling may be derived on the basis of a sensor signal, a measurement, a message from an external source, a maintenance or upgrade schedule, and/or a detected need for training.

The outputting of the at least one selected animated video instruction may be conditional on user confirmation or automatically output at the monitor.

For example, the controller may be configured to prompt the user for confirmation of outputting of the at least one selected animated video instruction, wherein the outputting of the at least one selected animated video instruction is subject to user confirmation. This may be advantageous for allowing a user to continue operation of the blood gas analyzer, e.g., for analysis of the blood sample, rather than having the at least one selected animated video instruction being output and optionally having to complete the illustrated handling of one or more user-accessible parts. Allowing a user to continue operation may be preferable in case of expert users, which are capable of responsibly deciding between, e.g., on the one hand following prompted instructions and on the other hand assessing the prompted instructions to be irrelevant for a certain analyte parameter measurement and then instead performing an urgent analyte parameter measurement. This may prevent that valuable time is lost before obtaining a result of the analysis of the blood sample.

Alternatively, the controller is configured to not allow the user to bypass the output of the at least one selected animated video instruction. In this case, blood sample analysis or other operation of the device may not be possible until the handling action, to which the animated video relates, has been completed. This may be preferable in case of non-expert users which cannot be expected to be capable of correctly assessing the consequences of omitting carrying out the handling action of the at least one selected animated video instruction, and where neglecting said handling may have adverse effects, e.g., damage of the blood gas analyzer, malfunction of the blood gas analyzer and/or imprecise or erroneous analyte parameter measurements.

The blood gas analyzer may comprise a sensor system for detecting the completion of steps to which the at least one selected animated video instruction relates, and wherein the controller is configured to progress outputting of the least one animated video instruction on the basis of the detected completion of completed steps. For example, the animated video instruction may omit outputting animated video instructions relating to completed steps and/or not progress to a subsequent step until the user has completed a current step. An advantage thereof may be increased safety and/or improved ease of use because the fit between information presented and the actual situation of the blood gas analyzer may be improved.

For instance, in the case that the user is experienced, he or she may be well aware that specific steps need to be performed, e.g. opening or closing a lid, removing a specific user-accessible part, etc. The user may therefore perform such steps promptly and without the need for guidance in the form of animated instructions. When the sensor system detects that such steps have already been performed, this is an indication that the user is experienced and does not require guidance with regard to these steps. Accordingly, there is no need to present the corresponding parts of the animated video instructions, and these parts are therefore omitted. Thus, the user will not waste time watching instructions which are not necessary, and the task can therefore be completed faster and without annoyance to the user. This may be very relevant, e.g. when the blood gas analyzer is positioned at a point-of-care, where staff may be busy and obtaining analysis results may be time critical.

In order to allow communication with an external device or network, the blood gas analyzer may comprise a communication interface for communicating with an external device or network. The communication interface may be unidirectional, either from the blood gas analyzer to the external device or network or vice versa, or bi-directional.

Communication from the blood gas analyzer to the external device or network may be advantageous for allowing information regarding status of the blood gas analyzer to be obtained externally, e.g., so as to communicate errors, order spare parts, etc. to a user or maintenance responsible person at a remote location. Communication from the external device or network to the blood gas analyzer may be advantageous for enabling an external party, to amend, such as update, one or more of the least two pre-stored sets of animated video instructions.

A bi-directional communication interface may enable both of the above advantages (associated with communication in either direction between the blood gas analyzer and the external device or network), e.g., to allow an external expert to obtain information regarding the status of the blood gas analyzer, which status may be prompting an update of a set of animated video instructions after which the external expert can update the set of video instructions and subsequently communicate the updated set of animated video instructions to the electronic memory of the blood gas analyzer.

A unidirectional communication interface may be advantageous, e.g., for prohibiting sensitive data, such as measurements results, being communicated from the blood gas analyzer or for prohibiting that an external party can alter settings in the blood gas analyzer.

A (local) user may, e.g. upon proper authorization, be allowed to amend (e.g., update) one or more of the at least one animated video instructions in the electronic memory of the blood gas analyzer, such as to optimize instructions provided to one or more users. Such option of locally amending the animated video instructions may dispense with a need of communication from an external device or network.

According to a second aspect, the invention provides a system for performing a measurement on analyte parameters in a blood sample comprising a blood gas analyzer according to the first aspect and a blood sample container. The system may in particular include the blood gas analyzer according to the first aspect of the invention, including any embodiment thereof disclosed herein. Accordingly, the remarks set forth above with reference to the first aspect of the invention are equally applicable in relation to the system of the second aspect of the invention.

According to a third aspect, the invention further provides a use of blood gas analyzer according to the first aspect for point-of-care (POC) measurement on analyte parameters in a blood sample. POC measurement is also referred to as ‘bed site’ measurement in the art. In the present context, the term ‘point-of-care measurement’ should be understood to mean measurements which are carried out in close proximity to a patient, i.e. measurements that are not carried out in a laboratory. Thus, according to this aspect, the user of the blood gas analyzer performs a measurement of a blood sample in a handheld blood sample container in the proximity of the patient, from whom the blood sample is taken, e.g. in the hospital room or ward accommodating the patient's bed, or in a nearby room of the same hospital department. In such use, to select at least one of a plurality of pre-stored sets of instructions in response to a signal as defined above provides a way of presenting instructions to the user, which guides the user to achieve convenient and efficient handling of the at least one user-accessible part is of particular importance.

The use may in particular include use of the blood gas analyzer according to the first aspect of the invention, including any embodiment thereof disclosed herein. Accordingly, the remarks set forth above with reference to the first aspect of the invention are equally applicable in relation to the use according to the third aspect of the invention.

### BRIEF DESCRIPTION OF THE DRAWINGS

The invention will now be described in further details with reference to the accompanying drawings, in which

FIG. 1 is a schematic diagram of a blood gas analyzer according to an embodiment of the invention,

FIG. 2 is a side-view of a blood gas analyzer according to an embodiment of the invention,

FIGS. 3-8 illustrate a sequence of steps included in instructions for a user's performance of a handling action of one or more user-accessible parts of the blood gas analyzer.

### DETAILED DESCRIPTION OF THE DRAWINGS

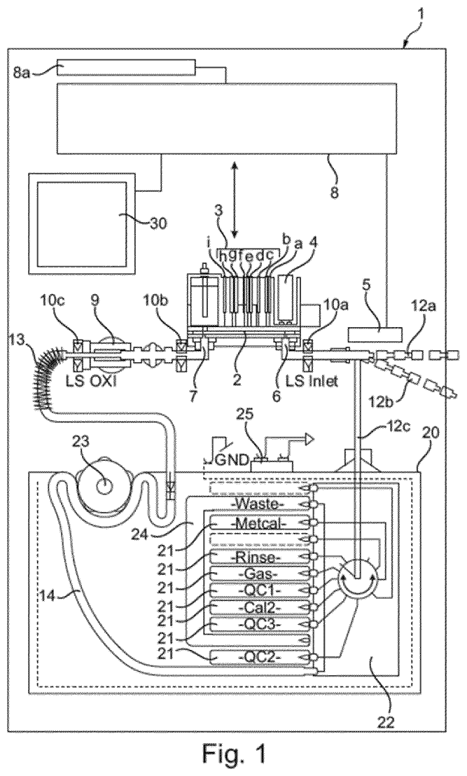

FIG. 1 is a schematic diagram of a blood gas analyzer 1 having a controller 8, one or more analyte sensors 3(a-i) and 4, a measurement chamber 2, and fluid handling infrastructure 20. For performing measurements, the user may provide a blood sample at an inlet structure 12 a/b of the blood gas analyzer 1 using a handheld blood sample container 100 (see FIGS. 3-10). When connecting the handheld blood sample container to the inlet structure 12a/b, a sensor system 5 detects a presence, a position and/or an orientation of the handheld blood sample container. A sensor signal of the detected presence, position and/or orientation of the handheld blood sample is provided from the sensor system 5 to the controller 8. The controller 8 assesses whether the detected sensor signal indicates if the handheld blood sample container 100 is in fluid communication with an aspiration point.

The blood sample is transferred from the aspiration point through an inlet 6 to the measurement chamber 2 comprising a plurality of analyte sensors 3 and 4. The analyte sensors 3 and 4 are arranged to provide measurements on analyte parameters in the blood sample. The analyte sensors 3 and 4 generate signals that are representative of a physical parameter for the respective analyte and provide the signals to the controller 8. The controller 8 is adapted to receive and process signals from the analyte sensors 3 and 4 and present the processed signals as output to the user at the monitor 30. The fluid handling infrastructure 20 includes a number of reservoirs 21 pre-filled with process liquids for, e.g., rinsing/wash-out, calibration and quality control tasks, as indicated in FIG. 1. The exact composition of a given process liquid may be stored in a chip 25. The process liquid for a given process step may be selected by a fluid selector valve 22, and via feed line 12c transferred through the aspiration point to the measurement chamber 2. Correct filling of the measurement chamber 2 is monitored by means of liquid sensors 10(a-c), located, e.g., at the aspiration point, at an outlet of the measurement chamber 2, and after a measurement device 9. The fluid flow through the blood gas analyzer 1 is driven by a pump 23, connected to a measurement device 9 via a fluid line 13. The discharged process fluids are transported through fluid line 14 to a waste reservoir 24.

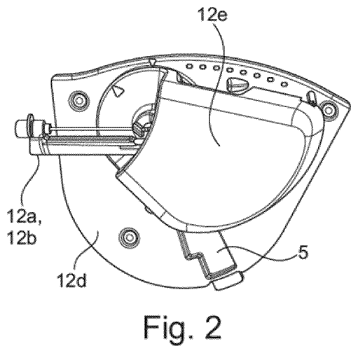

FIG. 2 is a side-view of a blood gas analyzer inlet module. The blood gas analyzer of FIG. 2 may be in accordance with the blood gas analyzer according to the FIG. 1. As shown, the inlet structure of the blood gas analyzer may include a stationary part 12d and an inlet module 12e.

FIGS. 3-8 illustrate a sequence of steps included in instructions for a user's performance of a handling action of one or more user-accessible parts of the blood gas analyzer. More particularly, FIGS. 3-8 specifically show replacement of an inlet gasket with holder.

FIG. 3 shows the blood gas analyzer 1 with inlet structure 12 a/b and monitor 30. For the purpose of presenting instructions to the user 102 in order to guide the user to convenient and efficient handling of the at least one user-accessible part (in this case the replacement of an inlet gasket with holder), a selected pre-stored set of animated video instructions is output to the user 102 via the monitor 30. In FIG. 3 the animated video instructions shows a hand gripping the inlet gasket with holder to be replaced.

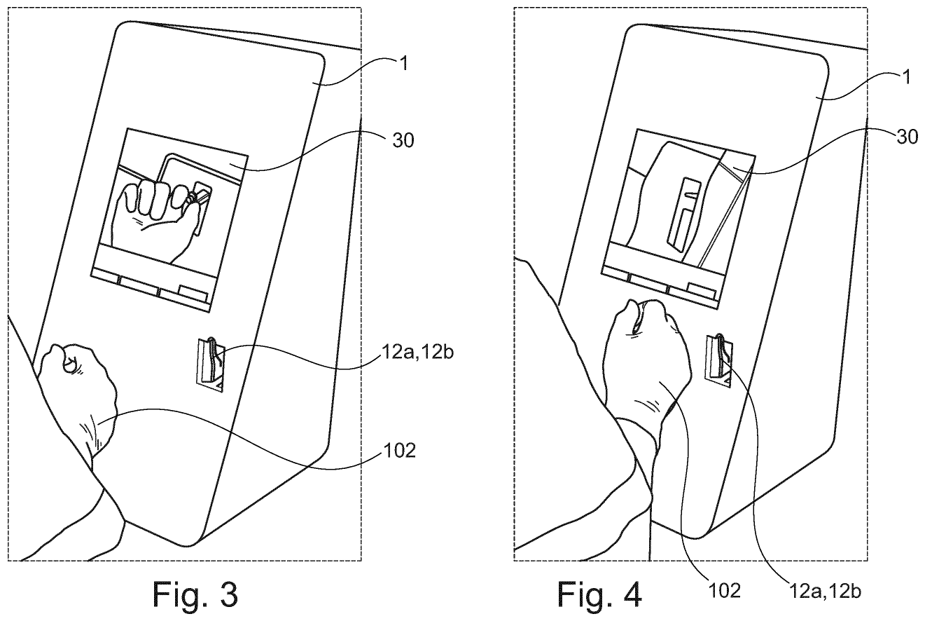

FIG. 4 depicts a situation subsequent to the user having removed the inlet gasket with holder to be replaced, where the animated video instructions has progressed so that the animated video instructions being output at the monitor 30 show no inlet gasket with holder at the position of the blood gas analyzer where the inlet gasket with holder to be replaced was previously positioned.

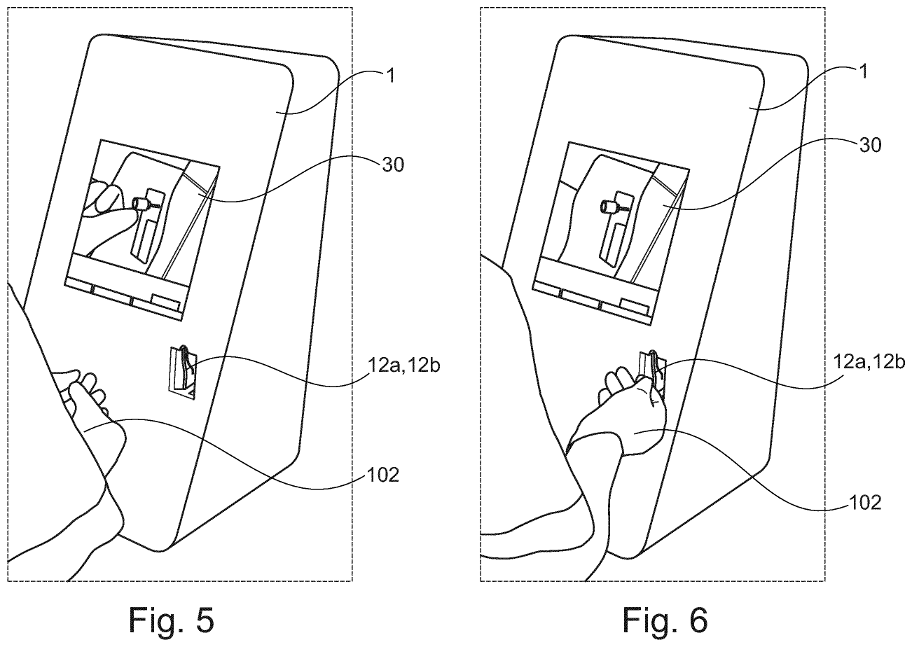

FIG. 5 shows the animated video instructions showing a hand holding a replacement inlet gasket with holder at the position of the blood gas analyzer where it is to be mounted.

The output of particular animated video instructions, such as these particular parts relating to a specific step to be carried out by the user (such as the animated video instructions depicted in FIGS. 4-5 relating to mounting of the replacement inlet gasket with holder at the empty position of the removed inlet gasket with holder), may be a result of a signal indicating the completion by the user of the removal of the inlet gasket with holder to be removed and/or a signal indicating that a replacement inlet gasket with holder has not yet been installed. For example, both of the above mentioned signals would enable the controller to arrange that the animated video instructions correspond to the actual situation, i.e., showing only the relevant step, such as showing the actual situation (presently no inlet gasket with holder at the position where the replacement inlet gasket with holder is to be mounted, cf., the animated video instruction depicted in the monitor in FIG. 4) and the next step to be carried out (mounting of the replacement inlet gasket with holder, cf., the animated video instruction depicted in the monitor in FIG. 5). This may be advantageous as the user might then not need to spend time (or be confused by) animated video instructions regarding the (completed) removal of the inlet gasket with holder to be replaced (cf., the animated video instruction depicted in the monitor in FIG. 3) and/or regarding not yet relevant steps.

FIG. 6 shows the user following the animated video instruction on the monitor 30 (cf., FIG. 5) and more particularly the hand of the user 102 placing the replacement inlet gasket and holder at the appropriate position of the blood gas analyzer in accordance with the animated video instructions depicted at the monitor 30.

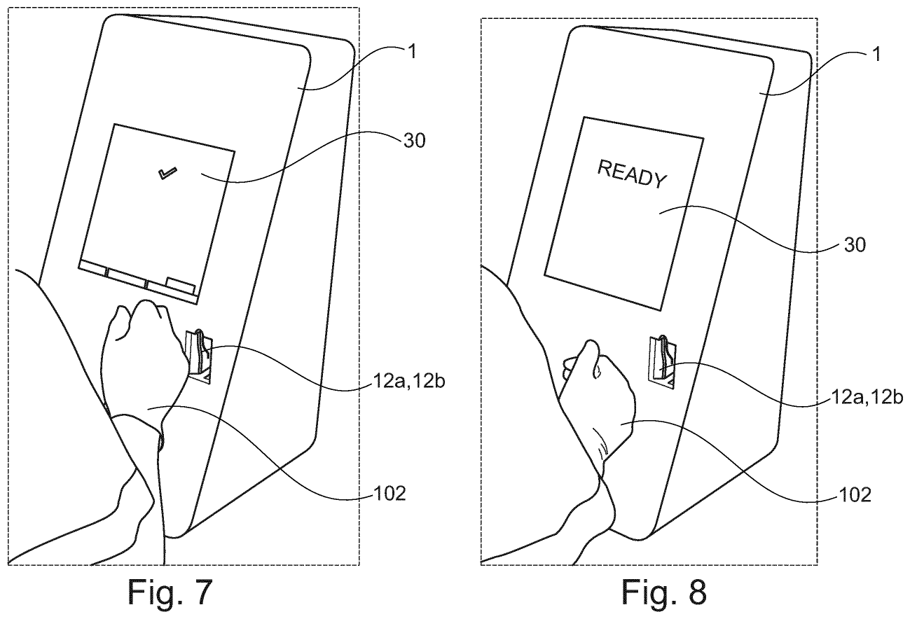

FIG. 7 is an illustration of the monitor outputting a visual indication to the user 102 that the handling is completed.

FIG. 8 is an illustration of the monitor outputting a visual indication to the user 102 that the blood gas analyzer is ready for performing a measurement on analyte parameters in a blood sample, such as a whole blood sample, aspirated into the blood gas analyzer from a blood sample container.

While the steps and animated video instructions depicted in FIGS. 3-8 depict a specific workflow relating to replacement of inlet gasket with holder, it is understood that the steps and animated video instructions could reflect other workflows to be carried out by the user in respect of the handling of other actions, i.e. another step or another plurality of sequential steps. For example, animated video instructions illustrating the user's correct handling, such as a step or a sequence of steps, could relate to replacement of other parts (such as an inlet module, an inlet connecter gasket), cleaning (such cleaning of an inlet gasket with holder and inlet area or such as back flush flow, where a cleaning liquid is flushed backwards through the system and out of the inlet through which blood samples during normal use are aspirated), or other actions.

1. A blood gas analyzer for performing a measurement on analyte parameters in a blood sample aspirated into the blood gas analyzer from a blood sample container, the blood gas analyzer comprising a plurality of user-accessible parts, a controller, and:
an aspiration system for aspirating the blood sample from the blood sample container, the aspiration system comprising an inlet structure for connecting to the blood sample container;
a monitor for outputting instructions to a user of the blood gas analyzer for the user's handling of one or more of user-accessible parts of the blood gas analyzer;
an electronic memory comprising at least two pre-stored sets of animated video instructions, each of the pre-stored sets of animated video instructions illustrating the user's correct handling of at least one of the user-accessible parts of the blood gas analyzer;
wherein the controller is configured to:
receive a signal indicative of at least one of:
a user input;
a presence, a position, and/or an orientation of the user-accessible parts, and
at least one projected handling action of at least one of the user-accessible parts of the blood gas analyzer; and
in response to the signal, determine if one of the animated video instructions is to be presented to the user, and in the affirmative:
select at least one of the at least two pre-stored sets of animated video instructions on the basis of the signal; and
output the at least one selected animated video instruction at the monitor.

2. The blood gas analyzer according to claim 1, wherein the user-accessible parts of the blood gas analyzer comprises at least one of:
a solution pack,
an inlet module,
a sensor cassette,
an inlet probe of the inlet structure,
an inlet connector gasket, and
an inlet gasket with a holder therefor.

3. The blood gas analyzer according to claim 1, wherein said signal is indicative of a need or a user desire to conduct at least one of:
a manual flush for removing clots in a conduit of the blood gas analyzer,
replacement or installation of the solution pack,
replacement or installation of the sensor cassette,
replacement of the inlet probe,
replacement of the inlet connector gasket,
replacement of the inlet gasket with holder, and
replacement of the inlet module.

4. The blood gas analyzer according to claim 1, wherein each of the animated video instructions illustrates a sequence of steps of the user's correct handling of the at least one user-accessible part.

5. The blood gas analyzer according to claim 4, wherein:
the signal indicative of the projected handling action includes an indication if one or more of said steps have been completed; and
the controller is configured to carry out the selection of the animated video instructions in such a manner that those one or more of the animated video instructions or those one or more parts thereof associated with the completed steps are omitted from outputting at the monitor.

6. The blood gas analyzer according to claim 1, wherein each of the pre-stored sets of animated video instructions comprises a simulation of a haptic feedback during the user's handling of the at least one of the user-accessible parts.

7. The blood gas analyzer according to claim 1, wherein the aspiration system comprises an aspiration point for establishing a fluid flow communication with the blood sample container, and wherein the blood gas analyzer further comprises a detection structure for detecting a presence of the blood sample container at the aspiration point.

8. The blood gas analyzer according to claim 7, wherein the controller is configured to:
deny the performance of analyte parameter measurements if it is determined that one or more of the at least one projected handling actions or one more steps thereof is due; or
prompt for a user confirmation of the performance of analyte parameter measurements if it is determined that one or more of the at least one projected handling action is due.

9. The blood gas analyzer according to claim 1, wherein the signal indicative of the projected handling is derived on the basis of a sensor signal, a measurement, a message from an external source, a maintenance or upgrade schedule, and/or a detected need for training.

10. The blood gas analyzer according to claim 1, wherein the controller is configured to prompt the user for confirmation of outputting of the at least one selected animated video instruction, and wherein the outputting of the at least one selected animated video instruction is subject to user confirmation.

11. The blood gas analyzer according to claim 1, wherein the controller is configured to not allow the user to bypass the output of the at least one selected animated video instruction.

12. The blood gas analyzer according to claim 1, further comprising a sensor system for detecting the completion of steps to which the at least one selected animated video instruction relates, and wherein the controller is configured to progress outputting of the least one animated video instruction on the basis of the detected completion of completed steps.

13. The blood gas analyzer according to claim 1, further comprising a communication interface for communicating with an external device or network.

14. A system for performing a measurement on analyte parameters in a blood sample comprising a blood gas analyzer according to claim 1 and a blood sample container.

15. A method of performing point-of-care (POC) measurement on analyte parameters in a blood sample, the method comprising:
obtaining the blood sample from a patient;
aspirating blood from the blood sample into the blood gas analyzer of claim 1; and
measuring analyte parameters in said blood.
